(19) United States
(12) Patent Application Publication
(10) Pub. No.: US 2003/0068322 A1
(43) Pub. Date: Apr. 10, 2003

(54) Methods of antibody-directed enzyme-prodrug therapy

(75) Inventor: Hans John Hansen, Little Egg Harbor, NJ (US)

Correspondence Address: FOLEY AND LARDNER, SUITE 500, 3000 K STREET NW, WASHINGTON, DC, 20007, US

(73) Assignee: IMMUNOMEDICS, INC.

(21) Appl. No.: 10/287,543

(22) Filed: Nov. 5, 2002

Related U.S. Application Data
(63) Continuation of application No. 09/216,609, filed on Dec. 21, 1998.
Application No. 09/216,609 is a continuation-in-part of application No. 08/445,110, filed on May 19, 1995, now Pat. No. 5,851,527.
Application No. 08/445,110 is a continuation of application No. 07/182,623, filed on Apr. 18, 1988, now abandoned.

(51) Int. Cl. A61K 39/395; G01N 33/574; C07K 16/46

(52) U.S. Cl. 424/178.1; 424/131.1; 435/7.23; 530/391.1

(57) ABSTRACT

A method for targeting a therapeutic agent to a target site in a patient is provided. The method comprises (a) administering to the patient an effective amount for targeting of at least one multispecific targeting protein comprising at least one first binding site which specifically binds to a substance produced by or associated with the target site and present at the target site and at least one second binding site which specifically binds to an epitope on an enzyme, wherein binding between the targeting protein and the enzyme does not interfere with enzyme activity; (b) administering to the patient an effective amount for enzyme activity of the enzyme, such that the targeting protein binds the enzyme to form a targeting protein-enzyme conjugate in situ; (c) administering to the patient at least one serum-soluble prodrug composition, wherein the enzyme administered in step (b) acts on the prodrug to release a therapeutic agent that is less soluble in serum than the prodrug, and wherein the therapeutic agent partitions out at the target site such that it accretes at the target site to a greater extent than would the prodrug, thereby providing therapeutic agent at the target site. One ore more optional clearing agents may be administered before or after the enzyme. In an alternative embodiment, the targeting protein-enzyme conjugate is made in vitro and administered in a single step. Injectable preparations and kits for carrying out the methods also are provided.

### RELATED APPLICATIONS

This application is a continuation-in-part of U.S. application Ser. No. 08,445,110, filed May 19, 1995 (now U.S. Pat. No. 5,851,527), which is a continuation of U.S. application Ser. No. 07/182,623, filed Apr. 18, 1988, now abandoned. The entire contents of these patent applications, including their specifications, claims, and abstracts, are incorporated herein by reference, as are all of the patents, patent applications and publications cited herein below.

### BACKGROUND OF THE INVENTION

1. Field of the Invention

The present invention relates to methods for targeting a therapeutic agent to a target site using an targeting protein-enzyme conjugate and a separate soluble substrate-therapeutic agent conjugate, wherein the targeted enzyme catalyzes the release of the therapeutic agent from the soluble substrate-therapeutic agent, thereby accumulating therapeutic agent at the target site for effective therapy. The foregoing method is useful for targeting any type of agent to a site to which a targeting protein (i.e., an antibody or antibody fragment) can selectively bind, including use for the therapy of tumors, infectious lesions, fibrin clots, myocardial infarctions, non-cancerous cells, damaged normal cells, atherosclerotic plaque, and lymphocyte autoreactive clones.

2. Description of Related Art

Therapy often requires a high absolute accretion of the therapeutic agent at the malignant site to permit a long duration of uptake, binding, or exposure. High dosages result in high background levels, however, which may result in undesired harm to healthy cells and tissue. Attempts to overcome the problems of non-specific delivery of cytotoxic therapeutic agents have involved the use of antibodies or antibody fragments coupled with therapeutic agents. These “direct targeting” methods are useful for treating tumors, infectious lesions, fibrin clots, myocardial infarctions, non-cancerous cells, damaged normal cells, atherosclerotic plaques, and lymphocytes.

A major obstacle encountered in such methods has been the difficulty of loading the antibody with a sufficient amount of therapeutic agent. Overloading the antibody with therapeutic agent may cause the patient to reject and destroy the antibody conjugate. Furthermore, conjugation of a large number of drug molecules to an antibody eventually reduces the antibody's immunoreactivity, with reduced immunoreactivity observed, for example, if more than about ten drug molecules are conjugated to an antibody.

To obviate this problem, drugs have been conjugated to a polymeric carrier, which in turn is conjugated to an antibody. This approach permits the delivery of larger numbers of drug molecules to the target site, but also raises other problems, as discussed below.

The use of polylysine as a polymer carrier was reported by Ryser et al., Proc. Natl. Acad. Sci. USA, 75:3867-70 (1978). These authors found that only about 13 methotrexate (MTX) molecules per carrier could be loaded and even then immunoreactivity was poor. In addition, the high amine content of the polymer, largely in the form of charged ammonium groups, caused the conjugate to stick to normal cells and vitiated the selectivity of the cytotoxic effect.

Rowland, U.S. Pat. No. 4,046,722, discloses an antibody conjugate wherein a plurality of molecules of a cytotoxic agent are covalently bound to a polymer carrier having a molecular weight of from 5,000-500,000, and the loaded carrier is covalently bound to an antibody by random attachment to pendant amine or carboxyl groups. Ghose et al., J. Natl. Cancer Inst., 61:657-76 (1978), discloses other antibody-linked cytotoxic agents useful for cancer therapy. Shih et al., U.S. Pat. No. 4,699,784, discloses site-specific attachment of a methotrexate-loaded aminodextran to an antibody.

Targeted neutron-activated radiotherapy is described, for example, in Goldenberg et al., Proc. Natl. Acad. Sci. USA, 81:560 (1984); Hawthorne et al., J. Med. Chem., 15:449 (1972); and in Goldenberg, U.S. Pat. Nos. 4,332,647, 4,348,376, 4,361,544, 4,468,457, 4,444,744, 4,460,459, 4,460,561, 4,818,709 and 4,624,846 the disclosures of all of which are incorporated herein by reference in their entireties. The aforementioned references disclose, inter alia, methods of incorporating Boron-10-containing addends into antibody conjugates using, e.g., a carborane coupled to an antibody (e.g., linked to a phenyldiazonium ion). These methods permit incorporation of a relatively low number of Boron-10 atoms. Typically, between 10 and 120 B-10 atoms have been attached to IgG using the carborane-phenyldiazonium conjugation procedure before the immunoreactivity and yield of recovered product becomes unacceptably low. It is desirable to be able to target a large number of B-10 atoms to a tumor site for effective therapy.

A need therefore continues to exist for a method of antibody-directed targeting where it is possible to deposit an effective amount of a therapeutic or diagnostic agent at a target site without overloading the targeting antibody with the agent and thereby losing immunoreactivity and/or inducing an immunogenic response.

### SUMMARY OF THE INVENTION

It is an object of the present invention to provide improved antibody-directed therapy methods that deliver an effective amount of therapeutic agent to the target site while minimizing cytotoxicity to non-targeted cells and tissue. It is another object of the present invention to provide compositions and kits for carrying out these methods.

Therefore, in accordance with one aspect of the invention, there is provided a method for targeting a therapeutic agent to a target site in a patient, comprising: (a) administering to the patient an effective amount for targeting of at least one multispecific targeting protein comprising at least one first binding site which specifically binds to a substance produced by or associated with the target site and present at the target site and at least one second binding site which specifically binds to an epitope on an enzyme, wherein binding between the targeting protein and the enzyme does not interfere with enzyme activity; (b) optionally, administering to the patient an amount effective for clearance of a first clearing composition comprising a clearing agent which clears non-localized targeting protein from circulation; (c) administering to the patient an effective amount for enzyme activity of the enzyme, such that the targeting protein binds the enzyme to form a targeting protein-enzyme conjugate in situ; (d) optionally, administering to the patient an amount effective for clearance of a second clearing composition comprising a clearing agent which clears non-localized targeting protein, non-localized enzyme, or non-localized targeting protein-enzyme conjugate from circulation; (e) administering to the patient at least one serum-soluble prodrug composition, wherein the enzyme administered in step (c) acts on the prodrug to release a therapeutic agent that is less soluble in serum than the prodrug, and wherein the therapeutic agent partitions out at the target site such that it accretes at the target site to a greater extent than would the prodrug, thereby providing therapeutic agent at the target site.

In accordance with specific embodiments of the invention, the targeting protein comprises a conjugate of at least one antibody, antibody fragment, or antibody subfragment which provides the first binding site and at least one antibody, antibody fragment or antibody subfragment which provides the second binding site, or it comprises a fusion protein comprising the first and second binding sites, or it comprises a covalent conjugate of at least two single chain antibodies, wherein at least one single chain antibody provides the first binding site and at least one single chain antibody provides the second binding site.

In accordance with other specific embodiments of the invention, step (a) comprises administering at least two different targeting proteins each of which comprise a first binding site which specifically binds to a different substance produced by or associated with the target site and present at the target site, and/or step (a) comprises administering at least two different targeting proteins each of which comprise a second binding site which specifically binds to a different enzyme, and step (c) comprises administering the different enzymes.

In accordance with other specific embodiments of the invention, the targeting protein comprises at least two first binding sites which specifically bind to the same or different epitopes of the same or different substance produced by or associated with the target site and present at the target site, and/or the targeting protein comprises at least two second binding sites which specifically bind to different enzymes, and step (c) comprises administering the different enzymes.

In accordance with one embodiment of the invention, the targeting protein, the enzyme, or both, is labeled with a detectable label such that step (c) results in the in situ formation of a labeled targeting protein-enzyme conjugate, and the method further comprises, prior to step (e), detecting the detectable label to determine the location of the targeting protein-enzyme conjugate.

In accordance with another embodiment of the invention, the targeting protein, the enzyme, or both, comprises a therapeutic agent such that step (c) results in the in situ formation of a targeting protein-enzyme conjugate comprising a therapeutic agent.

In accordance with another aspect of the invention, the therapeutic agent of the prodrug comprises a boron addend, and the method further comprises, after step (e), irradiating the boron.

The invention also provides a method for targeting a therapeutic agent to a target site in a patient, comprising: (a) administering to the patient at least one targeting composition comprising at least one targeting protein-enzyme conjugate comprising a binding site which specifically binds to a substance produced by or associated with the target site and present at the target site, and allowing the conjugate to localize at the target site; (b) optionally, administering to the patient a clearing composition comprising a clearing agent which clears non-localized targeting protein-enzyme conjugate from circulation; and (c) administering to the patient at least one serum-soluble prodrug composition, wherein the enzyme- of the targeting protein-enzyme conjugate administered in step (a) acts on the prodrug to release a therapeutic agent that is less soluble in serum than the prodrug, and wherein the therapeutic agent partitions out at the target site such that it accretes at the target site to a greater extent than would the prodrug, thereby providing therapeutic agent at the target site.

In accordance with one embodiment of this aspect of the invention, the targeting protein-enzyme conjugate comprises a fusion protein of the targeting protein and the enzyme.

Kits and sterile, injectable preparations for carrying out these methods also are provided.

### DETAILED DESCRIPTION OF THE INVENTION

Antibody-directed enzyme-prodrug therapy (ADEPT) delivers a nontoxic antibody-enzyme conjugate (or, more generally, a targeting protein-enzyme conjugate) to the target site, and then delivers a nontoxic prodrug conjugate that is converted to its active, toxic state by enzyme already present at the target site. ADEPT offers the significant promise of increasing by several orders of magnitude the amount of drug delivered to a target site compared to the amount delivered when the drug is directly conjugated to the antibody. ADEPT also offers the possibility of overcoming the limitation of the localization of only a small amount of antibody at the target site that is observed in clinical applications of direct targeting methods. The increased efficacy obtainable with ADEPT is attributable at least in part to the power of the enzyme to generate amplified amounts of active drug at the target site.

The present invention provides improved ADEPT methods. In accordance with one embodiment of the invention, the targeting protein-enzyme conjugate is formed in situ by administering a multivalent targeting protein having at least one first binding site specific for the target site and at least one second binding site specific for the enzyme, and then administering the enzyme.

In accordance with another embodiment of the invention, the prodrug is a substrate-therapeutic agent conjugate that, when acted upon by the enzyme of the targeting protein-enzyme conjugate, releases the therapeutic agent. (The substrate of this conjugate is a substrate for the enzyme). In accordance with one aspect of this embodiment, the substrate-therapeutic agent conjugate is soluble in the route of administration (i.e., serum), to facilitate administration and transport to the target site. The therapeutic agent that is released by the enzyme need not be soluble, and in accordance with one aspect of this embodiment, the therapeutic agent is less soluble in serum than the substrate-therapeutic agent. Additionally, in one embodiment of the invention, the therapeutic agent has a more favorable partition coefficient for the target site than the prodrug. In accordance with this embodiment, therefore, as the soluble substrate-therapeutic agent conjugate comes into contact with pretargeted enzyme, insoluble therapeutic agent is released and partitions favorably to the target. This partitioning out of the therapeutic agent after being acted upon by the targeted enzyme permits the drug to selectively accrete at the target site.

The invention is not limited to these prodrugs, however. As discussed in more detail below, any compound that can be acted on by an enzyme to form an active therapeutic agent may be useful as a prodrug in accordance with the present invention.

In accordance with another embodiment of the present invention, the targeting protein-enzyme conjugate is labeled with a detectable label. This permits the practitioner to determine the location of the targeting protein-enzyme conjugate, and thus determine when the conjugate has localized at the target site, and when non-localized conjugate has been removed from circulation. This information permits the practitioner to optimize the timing of the administration of any clearing agent and the prodrug.

In accordance with another embodiment of the invention, more than one targeting protein and/or more than one enzyme is used. For example, the targeting protein may comprise a conjugate of different antibodies or antibody fragments, the targeting protein-enzyme conjugate may comprise one targeting protein conjugated to two or more enzymes, and mixtures of targeting protein-enzyme conjugates comprising the same or different targeting proteins conjugated to the same or different enzymes may be used.

In accordance with another embodiment of the invention, a therapeutic agent is conjugated to the targeting protein-enzyme conjugate. This embodiment enhances the therapy by providing two different therapeutic agents to the target site.

In accordance with yet another aspect of the present invention, a clearing agent is administered after the targeting protein-enzyme conjugate is administered or, when the conjugate is formed in vivo, after the targeting protein is administered. Advantageously, the clearing agent is anti-idiotypic to the targeting protein. Anti-idiotypic antibodies, such as monoclonal antibodies, advantageously are used. Anti-idiotypic clearing agents bind to the same site of the targeting protein (i.e., the antibody or antibody fragment) that binds to the target site; accordingly, anti-idiotypic clearing agents will bind and remove only circulating targeting protein or targeting protein-enzyme conjugate, and will not competitively remove targeting protein or targeting protein-enzyme conjugate already localized at the target site. The use of an anti-idiotypic clearing agent therefore avoids the problem of undesired clearance that may be observed with non-anti-idiotypic clearing agents. Moreover, because the anti-idiotypic clearing agent does not bind to the enzyme moiety of the targeting protein-enzyme conjugate, it will not inactivate enzyme present at the target site. In accordance another aspect of this embodiment, the clearing agent is conjugated with sugar moieties to aid its clearance by the hepatic system.

Specific aspects of the invention are described in detail below.

Targeting Proteins

In accordance with the present invention, the targeting, protein of the targeting protein-enzyme conjugate is any targeting protein that specifically binds to a substance produced by or associated with the target site, and present at the target site, so that the targeting protein is effective for targeting the targeting protein-enzyme conjugate to the target site. As used herein, the term “targeting protein” includes antibody species such as antibodies, antibody fragments, multivalent antibody species, such as conjugates or fusion proteins of two or more antibodies and conjugates or fusion proteins of two or more antibody fragments, as well as non-antibody targeting proteins. Unless otherwise indicated, the terms “antibody” and “antibodies” as used below refer to any targeting protein.

The target sites can be, but are not limited to, cancers, infectious and parasitic lesions, fibrin clots, myocardial infarctions, atherosclerotic plaques, damaged normal cells, non-cancerous cells and lymphocyte autoreactive clones.

Many antibodies are known which specifically bind marker substances produced by or associated with tumors or infectious lesions, and present at tumors or infectious lesions, including viral, bacterial, fungal and parasitic infections, and antigens and products associated with such microorganisms. See, e.g., Hansen et al., U.S. Pat. No. 3,927,193, and Goldenberg U.S. Pat. Nos. 4,331,647, 4348,376, 4,361,544, 4,468,457, 4,444,744, 4,460,459, 4,460,561, 4,818,708, and 4,624,846, the disclosures of all of which are incorporated herein by reference in their entireties. Antibodies against an antigen, e.g., a gastrointestinal, lung, breast, prostate, ovarian, testicular, brain or lymphatic tumor, a sarcoma or a melanoma also are within the scope of this invention.

Antibodies that target myocardial infarctions are disclosed in, e.g., Haber, U.S. Pat. No. 4,036,945, the disclosure of which is incorporated herein by reference in its entirety. Antibodies that target normal tissues and organs are disclosed in, e.g., U.S. Pat. No. 4,735,210, the disclosure of which is incorporated herein by reference in its entirety. Anti-fibrin antibodies are well known in the art, as are antibodies that bind to atherosclerotic plaques and to lymphocyte autoreactive clones.

Important cancer markers that can be targeted in accordance with the present invention include minimally internalizing, cell-surface markers such as prostrate-specific membrane antigen (PSMA, see Fair et al., The Prostrate 32: 140-48 (1997), the contents of which are incorporated herein by reference in their entirety), GM2, sTn, MUC1 (see Zhang et al., Clin. Cancer Res., 4(2): 295-302 (1998); Kandilogiannaki et al., Cancer Res. 58(19): 4324-32 (1998) , the contents of both of which are incorporated herein by reference in their entirety), GD3 (see Kuwana et al., Cancer Immunol. Immunother. (36(6): 373-80 (1993), the contents of which are incorporated herein by reference in their entirety), GD2 (see Yuki et al., J. Neurol. Sci. 149(2): 127-30 (1997), the contents of which are incorporated herein by reference in their entirety), CD20 (see Kaminski et al., N. Eng. J. Med. 329:459-65 (1993), the contents of which are incorporated herein by reference in their entirety), Her2/neu (see, e.g. Turner et al., Cancer Res. 58(23): 5466-72 (1998); Ross et al., Stem Cells 16(6): 413-28 (1998), the contents of which are incorporated herein by reference in their entirety) and Le(y). Other cancer markers that can be targeted in accordance with the present invention include insoluble mucin and proteins which accumulate at cancer sites as cells die by apoptosis and necrosis, such as CSAp (see Sharkey et al., Int. J. Cancer 72(3): 477-85 (1997), the contents of which are incorporated herein by reference in their entirety) and DNA histone (see Miller et al., Hybridoma 12(6): 689-98 (1993) the contents of which are incorporated herein by reference in their entirety).

In general, antibodies usually can be raised to any antigen, using any of the many conventional techniques now well known in the art. Any antibody to an antigen which is found in sufficient concentration at a site in the body of a mammal which is of therapeutic interest can be used to make an antibody-enzyme conjugate for use in the method of the invention. Preferred are antibodies having a specific immunoreactivity to a marker substance produced by or associated with the target site of at least 60% and a cross-reactivity to other antigens or non-targeted substances of less than 35%.

Antibodies can be whole immunoglobulin of any class, e.g., IgG, IgM, IgA, IgD, IgE, or hybrid antibodies with dual or multiple antigen or epitope specificities, or fragments, such as F(ab′)₂, F(ab)₂, Fab′, Fab and the like, including hybrid fragments. Preferred fragments are Fab′, F(ab′)₂, Fab, and F(ab)₂. Also useful are any subfragments retaining the hypervariable, antigen-binding region of an immunoglobulin and having a size similar to or smaller than a Fab′ fragment. This includes genetically engineered and/or recombinant proteins, whether single-chain or multiple-chain, which incorporate an antigen-binding site and otherwise function in vivo as targeting molecules in substantially the same way as natural immunoglobulin fragments. Such single-chain binding molecules are disclosed in U.S. Pat. No. 4,946,778, which is hereby incorporated herein by reference in its entirety.

Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin. The fragments may also be produced by genetic engineering.

Monoclonal antibodies (mAbs) are preferred because of their high specificities. They are readily prepared by what are now considered conventional procedures of immunizing mammals with immunogenic antigen preparation, fusing immune lymph or spleen cells with an immortal myeloma cell line, and isolating specific hybridoma clones. More unconventional methods of preparing monoclonal antibodies are not excluded from the scope of this invention.

Additionally or alternatively, humanized antibodies or fragments of humanized antibodies are used. Humanized monoclonal antibodies are produced by transferring mouse complementary determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, in Orlandi et al., Proc. Nat'l Acad. Sci. USA 86:3833 (1989). Techniques for producing humanized mAbs are described, for example, in Jones et al., Nature 321:522 (1986); Carter et al., Proc. Nat'l Acad. Sci. USA 89:4285 (1992); Sandhu, Crit. Rev. Biotech. 12:437 (1992), and Singer et al., J. Immun. 150:2844 (1993). See also Shevitz et al., J. Nucl. Med. 35:112 (1994), describing hMN-14, a humanized an anti-CEA antibody having the human IgG₁/κ isotype. See also co-pending U.S. applications Ser. Nos. PCT WO/US97/04696, PCT WO/US96/14832, and PCT WO/US97/04196, the entire contents of which are incorporated herein by reference. It will be appreciated that newer techniques for production of monoclonals can also be used, e.g., interspecies monoclonals, chimeric (e.g., human/mouse) monoclonals, genetically engineered antibodies and the like. Use of those antibody production methods are well understood by those skilled in the art.

As used herein, “targeting proteins” include antiserum preparations, preferably affinity purified antiserum preparations, that have high immunoreactivity, e.g., a binding constant of at least about 10⁷ 1/mole, preferably at least about 10⁹ 1/mole, and which are immunospecific, (at least about 40%, preferably at least about 60%, more preferably about 70-95%), and have low cross-reactivity with other tissue antigens (not more than about 30%, preferably not more than about 15% and more preferably not more than about 5%). The antiserum can be affinity purified by conventional procedures, e.g., by binding antigen to a chromatographic column packing, e.g., Sephadex, passing the antiserum through the column, thereby retaining specific antibodies and separating out other immunoglobulin and contaminants, and then recovering purified antibodies by elution with a chaotropic agent, optionally followed by further purification.

It should be noted that mixtures of antibodies and immunoglobulin classes can be used, as can hybrid antibodies. Multispecific, including bispecific and hybrid, antibodies and antibody fragments are useful in the methods of the present invention, and are comprised of at least two different substantially monospecific antibodies or antibody fragments, wherein at least two of said antibodies or antibody fragments specifically bind to at least two different antigens produced by or associated with the target site or at least two different epitopes or molecules of a marker substance produced by or associated with the target site. For example, when targeting colon cancer, one antibody or antibody fragment may bind specifically to the CEA antigen, and another antibody or antibody fragment may bind specifically to colon-specific antigen p. Other examples of useful antibody combinations include LL1 and LL@ anti-lymphoma monoclonal antibodies; anti-CD22 and anti-CD20 anti-lymphoma monoclonal antibodies; anti-CD22 and anti-CD19 anti-lymphoma monoclonal antibodies; and anti-CD19 and anti-CD20 anti-lymphoma monoclonal antibodies. Combinations including RS7, RS11, MN-14, anti-HER2 and MA5 antibodies, which target different antigens on major solid tumors such as breast, prostate and lung tumors, also are useful in the present invention.

Multispecific antibodies and antibody fragments with dual specificities can be prepared analogously to the anti-tumor marker hybrids disclosed in U.S. Pat. No. 4,361,544. Other techniques for preparing hybrid antibodies are disclosed in, e.g., U.S. Pat. No. 4,474,893 and 4,479,895, and in Milstein et al., Immunol. Today, 5: 299 (1984). For example, bispecific antibodies can be made by a variety of conventional methods, e.g., disulfide cleavage and reformation of mixtures of whole IgG or, preferably F(ab′)₂ fragments, fusions of more than one hybridoma to form polyomas that produce antibodies having more than one specificity, and by genetic engineering. Bispecific antibodies have been prepared by oxidative recombination of Fab′ fragments resulting from reductive cleavage of different antibodies. This is advantageously carried out by mixing two different F(ab′)₂ fragments produced by pepsin digestion of two different antibodies, reductive cleavage to form a mixture of Fab′ fragments, followed by oxidative reformation of the disulfide linkages to produce a mixture of F(ab′)₂ fragments including bispecific antibodies containing a Fab′ portion specific to each of the original epitopes (i.e., two different tumor-associated antigens). General techniques for the preparation of multivalent antibodies are found, for example, in Nisonnoff et al., Arch Biochem. Biophys. 93: 470 (1961), Hammerling et al., J. Exp. Med. 128: 1461 (1968), and U.S. Pat. No. 4,331,647.

A more selective linkage can be achieved by using a heterobifunctional linker such as maleimide-hydroxysuccinimide ester. Reaction of the ester with an antibody or antibody fragment will derivatize amine groups on the antibody or antibody fragment, and the derivative can then be reacted with, e.g., an antibody Fab fragment having free sulfhydryl groups (or, a larger fragment or intact antibody with sulfhydryl groups appended thereto by, e.g., Traut's Reagent). Such a linker is less likely to crosslink groups in the same antibody and improves the selectivity of the linkage. See also U.S. Pat. No. 5,772,981, the entire contents of which are incorporated herein by reference, which describes thiolation reagents and methods.

It is advantageous to link the antibodies or fragments at sites remote from the antigen binding sites. This can be accomplished by, e.g., linkage to cleaved interchain sulfhydryl groups, as noted above. Another method involves reacting an antibody having an oxidized carbohydrate portion with another antibody which has at lease one free amine function. This results in an initial Schiff base (imine) linkage, which is preferably stabilized by reduction to a secondary amine, e.g., by borohydride reduction, to form the final product. Such site-specific linkages are disclosed, for small molecules, in U.S. Pat. No. 4,671,958, and for larger addends in U.S. Pat. No. 4,699,784. In F(ab′)₂-Fab′ conjugates, the monomeric species is linked via a site remote to the antigen binding site while the F(ab′)₂ portion maintains its divalency.

Hydrazides and hydrazines also can be used for site-specific linkage via antibody aldehyde groups. Such reagents give more stable Schiff bases than amino groups. A particularly useful cross-linking agent would link the thiol groups of one protein (i.e., an antibody) to oxidized carbohydrate groups of another protein (i.e., another antibody). For example, an IgG or F(ab′)₂ fragment containing a carbohydrate can be oxidized with periodate and mixed with a commercially available cross-linking agent such as 4-(4-N-maleimidophenyl)butyric acid hydrazide (MPBH, Pierce Chemical Co., Rockford, Ill.). The maleimide-appended first protein is then mixed with the thiol-containing second protein to yield a bispecific antibody.

Alternatively, bispecific antibodies can be produced by fusing two hybridoma cell lines that produce anti-tumor-associated antigen mAb to two different tumor-associated antigens or to two different epitopes of a single tumor-associated antigen. Techniques for producing tetradomas are described, for example, by Milstein et al., Nature 305: 537 (1983) and Pohl et al., Int. J. Cancer 54: 418 (1993).

Bispecific antibodies also can be produced by genetic engineering. For example, plasmids containing DNA coding for variable domains of an anti-tumor-associated antigen mAb can be introduced into hybridomas that secrete antibodies to a different tumor-associated antigen or antibodies to a different epitope of the same tumor-associated antigen. The resulting “transfectomas” produce bispecific antibodies that bind different tumor-associated antigens. Alternatively, chimeric genes can be designed that encode both anti-tumor-associated antigen-binding domains. General techniques for producing bispecific antibodies by genetic engineering are described, for example, by Songsivilai et al., Biochem. Biophys. Res. Commun. 164: 271 (1989); Traunecker et al., EMBO J. 10: 3655 (1991); and Weiner et al., J. Immunol. 147: 4035 (1991).

A higher order multivalent, multispecific targeting protein can be obtained by adding various antibody components to a bispecific antibody, produced as above. For example, a bispecific antibody can be reacted with 2-iminothiolane to introduce one or more sulfhydryl groups for use in coupling the bispecific antibody to a further antibody derivative that binds to the same or a different epitope of the same tumor-associated antigen or to a different tumor-associated antigen, using the bis-maleimide activation procedure described above. These techniques for producing multivalent targeting proteins are well known to those of skill in the art. See, for example, U.S. Pat. No. 4,925,648, and Goldenberg, international publication No. WO 92/19273, which are incorporated by reference.

The antibodies, antibody fragments and antibody subfragments useful in the methods of the present invention may conjugated by a variety of methods known in the art. Many of these methods are disclosed in the above-referenced U.S. Patents and Patent Applications. See also Childs et al., J. Nuc. Med., 26: 293 (1985).

One antibody preferred for use in the present invention is MN-14, a second generation CEA-antibody that has ten times more affinity for CEA than the first generation version, NP-4. Hansen et al., Cancer, 71: 3478-85 (1993). MN-14 internalizes slowly, making it suitable for a pre-targeting approach. hMN-14 (humanized MN-14) also is preferred for use in the present invention.

While antibodies are preferred targeting proteins, other targeting proteins also may be used in the targeting protein-enzyme conjugate, such as those described in U.S. application Ser. Nos. 08/486,166 (abandoned) and 08/409,960, the entire contents of which are incorporated herein by reference. For example, proteins, peptides, polypeptides, glycoproteins, lipoproteins, phospholipids, oligonucleotides, steroids, alkaloids or the like, e.g., hormones, lymphokines, growth factors, cytokines, enzymes, immune modulators, receptor proteins, and antisense oligonucleotides which preferentially bind marker substances that are produced by or associated with the target site and present at the target site are useful in accordance with the present invention. Specific examples of such targeting proteins include VIP, LHRH, somatostatin, E-selectin, and their respective receptors. As used herein, therefore, the term antibody encompasses these non-antibody targeting proteins.

Enzymes

Enzymes useful in the present invention include any enzyme that can be conjugated to a targeting protein to form a soluble targeting protein-enzyme conjugate and that can convert a prodrug into an active therapeutic agent (drug). In one embodiment, a human enzyme is used, such as an enzyme normally found in human cells. If this enzyme normally is found in plasma, endogenous activity could be blocked with a blocking or clearing agent prior to administration of the targeting protein-enzyme conjugate. In another embodiment, the enzyme is a non-human or non-mammalian enzyme. In this embodiment, the immunogenicity of the enzyme optionally is reduced, for example, by conjugation to polyethylene glycol (PEG). In accordance with one embodiment, neither the enzyme nor an enzyme with similar substrate specificity is endogenous to the patient along the route of administration or biodistribution of the prodrug.

Proteases, glycosidases, esterases and the like are general types of enzymes that can be used in accordance with the present invention. Specific examples of suitable enzymes include, but are not limited to, glucuronidase, beta-glucosidase, beta-lactamase, cellulase, dextranase, fructase, aminopeptidase, lysozyme and carboxyesterase. Such enzymes are well known in the art and are readily available in purified form.

The enzyme is selected for its ability to convert the selected prodrug into its active drug form. For example, if the prodrug comprises a conjugate of dextran and a therapeutic agent, an appropriate enzyme would be dextranase. Similarly, cellulase could be used with a prodrug comprising a cellulose substrate, and glucuronidase could be used with a prodrug comprising a glucuronide.

In one embodiment, the enzyme is an alkaline phosphatase, β-lactamase, or carboxypeptidase G2. In a particularly preferred embodiment, the enzyme is carboxypeptidase G2 (CPG2). CPG2 is a folate depleting enzyme of bacterial origin. See Sherwood et al., Eur. J. Biochem. 148:447 (1985). Its use in activating prodrugs has been well documented. Its prodrug substrates are phenolic, benzoic or aniline derivatives of mustard. In terms of the cytoxicity of the active product, phenolic derivatives are more cytotoxic than benzoic derivatives. See Blakey et al., supra (1996). A comparative study of sixteen mustard derivatives identified one phenolic derivative, ZD2767P, as the best prodrug candidate based on physicochemical and biological characteristics. See Springer et al. J. Med. Chem. 38:5051-65 (1995).

Each enzyme molecule can transform many molecules or subunits of prodrug to many molecules of drug that, in accordance with one embodiment of the invention, will accrete at the target site due to favorable partition between the fluid bathing the target site and the tissue or other antigen-containing medium at the site itself. Thus, the enzyme amplifies the targeting capability of the targeting protein without the need to conjugate the agent to the targeting protein, and the agent accumulates at the target site and can effect its therapeutic action there.

Targeting Protein-Enzyme Conjugates

Targeting protein-enzyme conjugates can be made by any number of methods. In accordance with one embodiment of the invention, the targeting protein comprises a binding site specific for the-enzyme. For example, the targeting protein may be a conjugate comprising at least two antibodies or antibody fragments, at least one of which is specific for the target site and at least one of which is specific for the enzyme (or for a moiety linked to the enzyme), or it may comprise a covalent conjugate of two different single chain antibodies, one specific for the target site and one specific for the enzyme. Also useful in this embodiment are “diabodies,” which are single chain antibodies with two specificities, one for the target site and one for the enzyme. Diabodies are generally described in Knotermann et al., Immunotech. 3: 137-44 (1997), the entire contents of which are incorporated herein by reference. Targeting proteins useful in accordance with this embodiment can be made by the methods described above for making multivalent antibodies, including the conventional and recombinant methods. Preferably, the targeting protein does not bind to a site on the enzyme that interferes with enzyme activity.

In one specific example of this embodiment, an MN-14-F(ab′)₂ fragment is conjugated to an anti-DTPA-Fab′ fragment, and the enzyme, such as esterase, is conjugated to DTPA. Upon mixing in vitro or contact in vivo, an MN-14-F(ab′)₂-esterase conjugate is formed. The DTPA moiety can be conjugated to the enzyme via a linker, such that recognition by the anti-DTPA-Fab′ fragment is not affected. The substitution ratio of DTPA onto the enzyme can be readily controlled (and measured by quantitative radiolabelling) to a minimal level to preserve enzyme activity.

In another aspect of this embodiment, small molecular recognition units rather than full Fab′ fragments are used to link the targeting protein to the enzyme. Such non-covalent coupling of the targeting protein to, the enzyme is expected to give higher yields of desired product than covalent coupling methods.

In accordance with this embodiment, the targeting protein-enzyme conjugate can be made prior to administration by contacting the targeting protein with the enzyme, or can be made in situ by administering the targeting protein and enzyme sequentially, preferably permitting the targeting protein to localize at the target site and substantially clear from circulation before administering the enzyme. These methods obviate the need to covalently conjugate the enzyme to the targeting protein.

Alternatively, the targeting protein-enzyme conjugate may comprise a fusion protein of the targeting protein and the enzyme. Methods of making fusion proteins are well-known in the art, and include those referenced above with respect to targeting proteins. See also Haisma et al., Blood 92: 184-90 (1998).

In accordance with yet another alternative embodiment, the enzyme is covalently bound to the targeting protein, directly or through a linker moiety, through one or more non-functional groups on the targeting protein and/or the enzyme, such as amine, carboxyl, phenyl, thiol or hydroxyl groups. Conventional linkers can be used, such as diisiocyanates, diisothiocyanates, bis(hydroxysuccinimide) esters, carbodiimides, maleimide-hydroxysuccinimide esters, glutaraldehyde and the like, or hydrazines and hydrazides, such as MPBH.

A simple method for making a targeting protein-enzyme conjugate is to mix the targeting protein with the enzyme in the presence of glutaraldehyde to form the targeting protein-enzyme conjugate. The initial Schiff base linkages can be stabilized, e.g., by borohydride reduction to secondary amines. A diisothiocyanate or a carbodiimide can be used in place of glutaraldehyde.

More selective linkage can be achieved by using a heterobifunctional linker such as a maleimide-hydroxysuccinimide ester. Reaction of the latter with an enzyme will derivatize amine groups on the enzyme, and the derivative can then be reacted with, e.q., an antibody Fab fragment with free sulfhydryl groups (or a larger fragment or intact immunoglobulin with sulfhydryl groups appended thereto by, e.g., Traut's Reagent).

It is advantageous to link the enzyme to a site on the targeting protein remote from the antigen binding site. This can be accomplished by, e.g., linkage to cleaved interchain sulfhydryl groups, as noted above. Another method involves reacting an antibody whose carbohydrate portion has been oxidized with an enzyme which has at least one free amine function. This results in an initial Schiff base (imine) linkage, which is preferably stabilized by reduction to a secondary amine, e.g., by borohydride reduction, to form the final conjugate. See also the discussion above of the methods described in U.S. Pat. No. 5,772,981 and hydrazine and hydrazide linkages.

As discussed above, in accordance with one embodiment of the invention, the targeting protein-enzyme conjugate is labeled with, conjugated to, or adapted for conjugation to, a detectable label, such as a radioisotope or magnetic resonance image enhancing agent. If the targeting protein-enzyme conjugate is made in situ, the targeting protein, the enzyme, or both may be labeled with, conjugated to, or adapted for conjugation to, the detectable label, so that a labeled targeting protein-enzyme conjugate will be formed. This labeling permits the practitioner to monitor the localization and clearance of the targeting protein-enzyme conjugate, and optimize the timing of the clearing agent and prodrug administration steps.

In one aspect of this embodiment, the conjugate is labeled with a radiolabel, a fluorescent label or the like, that permits its detection and quantitation in body fluids, e.g., blood or urine, so that localization and clearance of the conjugate can be measured and/or inferred from the amount of conjugate measured in the bodily fluids.

Any conventional method of radiolabeling suitable for labeling proteins for in vivo use can be used to label the targeting protein-enzyme conjugates. For example, the conjugates can be directly labeled with I-131, I-123, or metallized with Tc-99m, Cu ions, or the like, by conventional techniques or by attaching a chelator for a radiometal or paramagnetic ion. Such chelators and their modes of attachment to antibodies are well known in the art, and described, for example, in the aforementioned Goldenberg patents and in Childs et al., J. Nuc. Med., 26:293 (1985). Positron emission (PET) agents also can be used, such as I-124, Tc-94m, Y-86, Zr-89, and Co-89. Likewise, magnetic resonance imaging (MRI) agents such as gadolinium, manganese, and iron can be used in this embodiment of the invention.

In one application of this embodiment, the time of maximum tumor uptake of the targeting protein-enzyme conjugate is determined by first determining the optimum enzyme dose, and then determining the time of maximum tumor uptake at this dose using a labeled targeting protein-enzyme conjugate. If a clearing agent is used, it is advantageously administered at this time. Otherwise, the prodrug advantageously is administered at this time.

As discussed above, in accordance with another embodiment of the invention, the targeting protein-enzyme conjugate comprises more than one antibody or antibody fragment and/or more than one enzyme. For example, it may be advantageous to bind a plurality of antibody fragments, e.g., Fab or F(ab′)₂ fragments, to a single enzyme to increase its binding affinity for the target site and to increase the targeting efficiency. The antibodies or antibody fragments may bind to the same or different epitopes on the same or different antigens at the target site. For example, when targeting colon cancer cells, one antibody or antibody fragment may bind specifically to the CEA antigen, and another antibody or antibody fragment may bind specifically to colon-specific antigen p. Other examples of useful antibody combinations include LL1 and LL2 anti-lymphoma monoclonal antibodies; anti-CD22 and anti-CD20 anti-lymphoma monoclonal antibodies; anti-CD22 and anti-CD19 anti-lymphoma monoclonal antibodies; and anti-CD19 and anti-CD20 anti-lymphoma monoclonal antibodies. Combinations including RS7, RS11, MN-14, anti-HER2 and MA5 antibodies, which target different antigens on major solid tumors such as breast, prostate and lung tumors, also are useful in the present invention.

Alternatively, if the enzyme is not too bulky, it may be useful to link a plurality of enzyme molecules to a single targeting protein to increase the amount of prodrug that is converted to drug per the amount of targeting protein administered. The enzymes may be the same or different. If different enzymes are used, then more than one prodrug may be administered, with each enzyme converting a different prodrug into its active drug form. In one embodiment, one enzyme converts the administered prodrug to its active form, and another enzyme converts a metabolite of the prodrug or active drug (i.e., a glucuronide) to the active form. For example, conjugates comprising glucuronidase and carboxyesterase could be used in conjunction with the prodrug CPT-11. The esterase cleaves CPT-11 to the active drug SN-38, and the glucuronidase converts any SN-38 glucuronide back to the active SN-38 form.

The size of the resulting conjugate may limit the number of targeting proteins and enzymes that are conjugated to a single conjugate, but conjugates of more than one targeting protein and/or more than one enzyme can be used as long as they can reach the target site and don't clear too fast. Conjugates under about 1,000,000 KDa are believed to be acceptable. Mixtures of different targeting protein-enzyme conjugates also can be used, such as conjugates comprising the same targeting protein but different enzymes or conjugates comprising different targeting proteins and different enzymes.

In accordance with another embodiment of the invention, the targeting protein-enzyme conjugate comprises at least one therapeutic agent different from the therapeutic agent of the prodrug. See PCT WO/US97/07395 the entire contents of which are incorporated herein by reference in their entirety. This embodiment of the invention addresses the problem of tumor heterogeneity by delivering at least two different therapeutic agents having different tumor-killing properties to the tumor sites. When the targeting protein-enzyme conjugate is made in situ, the targeting protein, the enzyme, or both, may comprise a therapeutic agent so that the resulting targeting protein-enzyme conjugate comprises a therapeutic agent.

The therapeutic agent may be selected from the group consisting of radionuclides, drugs, toxins, and boron addends. If the therapeutic agent of the targeting protein-enzyme conjugate and that of the prodrug both are radionuclides, then it is preferable that each of the radionuclides emit different levels of radiation. Preferably the therapeutic agent of the targeting protein-enzyme conjugate is selected from the group consisting of I-131, I-125 and At-211, and the therapeutic agent of the prodrug is selected from the group consisting of P-32, P-33, Sc-47, Cu-64, Cu-67, As-77, Y-90, Ph-105, Pd-109, Ag-111, I-125, Pr-143, Sm-153, Tb-161, Ho-166, Lu-177, Re-186, Re-188, Re-189, Ir-194, Au-199, Pb-212, and Bi-213.

In one aspect of this embodiment of the invention, the therapeutic agent of the targeting protein-enzyme conjugate is a radionuclide and the therapeutic agent of the prodrug is a drug, a toxin, or a boron addend. As described above, the radionuclide is selected from the group consisting of I-131, I-125 and At-211.

In a further aspect of this embodiment, the therapeutic agent of the targeting protein-enzyme conjugate is a drug or toxin and the therapeutic agent of the prodrug is a radionuclide or a boron addend. Suitable drugs include taxol, nitrogen mustards, ethylenimine derivatives, alkyl sulfonates, nitrosoureas, triazenes, folic acid analogs, pyrimidine analogs, purine analogs, vinca alkaloids, antibiotics, enzymes, platinum coordination complexes, substituted urea, methyl hydrazine derivatives, adrenocortical suppressants, hormones, antagonists, camptothecin, and endostatin. Suitable toxins include abrin, alpha toxin, diphtheria toxin, exotoxin, gelonin, pokeweed antiviral protein, ricin, saporin, and onconase.

In another aspect of this embodiment, the therapeutic agent of the targeting protein-enzyme conjugate is a drug or toxin and the therapeutic agent of the prodrug is a radionuclide or a boron addend.

Clearing Agents

In accordance with another embodiment of the invention, a clearing agent is used to effect the rapid clearance of circulating targeting protein-enzyme conjugate. The use of a clearing agent in accordance with the present invention reduces or eliminates problems associated with circulating targeting protein-enzyme conjugate. In clinical applications, only from 0.1 to 0.0001% of the injected dose of targeting protein-enzyme conjugate may actually localize at the target site. This leaves a large portion of the injected dose in circulation, where it may remain for several days, particularly when cdr-grafted antibodies are used as the targeting antibody. By removing non-localized targeting protein-enzyme conjugate from circulation, this embodiment of the present invention prevents the release of the therapeutic agent from the prodrug at sites distant from the target site. This in turn minimizes unwanted systemic toxicity due to the presence of the therapeutic agent in blood and other blood-rich tissues.

If the targeting protein-enzyme conjugate is made in situ, the clearing agent may be administered after the targeting protein has been administered and allowed to localize at the target site, and before or after the enzyme is administered. Advantageously, the clearing agent is administered before the enzyme is administered to remove circulating targeting protein and maximize the efficiency of the delivery of the enzyme to the target site. Alternatively, the clearing agent can be administered after the enzyme is administered to remove circulating targeting protein or circulation targeting protein-enzyme conjugate. In accordance with one embodiment, the clearing agent binds to the enzyme, preferable at a site that does not interfere with enzyme activity. In this embodiment, the clearing agent is administered after the enzyme has been administered and localized, and clears circulating enzyme and circulating targeting protein-enzyme conjugate. In one aspect of this embodiment, the clearing agent binds to the same epitope of the enzyme as the second binding site of the targeting protein. This specific embodiment minimizes the unwanted clearance of localized enzyme.

Although any clearing agent may be used in accordance with the present invention, anti-idiotypic clearing agents are advantageously used. Such clearing agents are anti-idiotypic to the binding site of the targeting protein that binds the target site. See, e.g., U.S. application Ser. No. 08/409,960. As used herein, the term “anti-idiotypic” means that the clearing agent specifically binds to the same site on the targeting protein that binds to the target site (the paratope or antigen-binding region).

Because the “anti-idiotypic” antibody, as used according to the present invention, functions as a clearing agent but not necessarily as an antigen mimic, it need not bind exclusively to the paratopic region of the idiotype/targeting protein but need only bind to a region on the targeting protein such that the paratope no longer can bind to the target antigen. Accordingly, the clearing agents also may be non-antibody species that bind to the antigen-binding site of the targeting protein. Preferred clearing agents of the present invention include anti-idiotypic monoclonal antibodies.

When the targeting protein-enzyme conjugate comprises not an antibody, but a non-antibody targeting species such as small peptides, steroids, hormones, cytokines, neurotransmitters, or other targeting species which preferentially bind marker substances that are produced by or associated with the target site and present at the target site, the clearing agent may comprise an antibody that specifically binds to the receptor-binding site of the targeting species, i.e., to a region that blocks binding to the target receptor. A non-antibody clearing agent also may be used which binds to the receptor-binding site of the non-antibody targeting species.

Because these clearing agents bind to the antigen-binding site of the targeting protein, they can only bind circulating targeting protein, and cannot bind targeting protein already bound to the target site. These clearing agents offer at least two distinct advantages over clearing agents currently used. First, they do not remove targeted targeting protein-enzyme conjugate from the target site, and second, they do not bind to or inactivate targeted enzyme.

Alternatively, the clearing agent may comprise an antibody, such as a monoclonal antibody, directed to an addend on the targeting protein-enzyme conjugate. For example, targeting protein-enzyme conjugate comprising DTPA, DOTA, biotin, histamine-succinate or fluorescein can be cleared with an antibody to DTPA, DOTA, biotin, histamine-succinate or fluorescein, respectively.

In one advantageous embodiment of the invention, the clearing agent is conjugated to sugar residues such as galactose residues which bind to the hepatic asialoglycoprotein receptor, whereby the clearing agent and clearing agent-targeting protein-enzyme complexes are rapidly recognized by liver hepatocytes. Use of such galactosylated clearing agents ensures near-total hepatocytic recognition and sequestration within minutes post-injection, generally substantially in a single pass through the liver.

The degree of sugar residue modification of the clearing agent determines the blood clearance rate. The number of sugar residues per molecule of clearing agent may be determined empirically for each specific clearing agent by routine methods well-known in the art. It is convenient to express the degree of glycosylation in terms of the percentage of lysine residues modified by addition of sugars. For anti-idiotypic antibody clearing agents, it has been found that modifying about 22% of the lysine residues does not provide significantly accelerated clearance of non-localized primary targeting conjugate, whereas modifying about 48% of the lysine residues greatly accelerate clearance and modifying about 76% or more of the lysine residues results in virtually total clearance from circulation in a single pass through the liver. This will generally be true for antibody fragments as well, although the percentages may vary somewhat. The degree of glycosylation to achieve substantially complete clearance in one pass is readily determined.

In accordance with one aspect of this embodiment of the invention, the administered prodrug is relatively non-toxic, of low molecular weight and structurally designed to have a desirable biological half-life. A targeting protein-enzyme conjugate is administered to a patient, and time is allowed for its accretion at the target site to reach a maximum level. This can readily be determined, for instance by using a radioactive label on the targeting protein-enzyme conjugate, as discussed above. Then, an antibody anti-idiotypic to the targeting protein is given. The anti-idiotypic antibody complexes and removes almost all circulating targeting protein-enzyme conjugate within minutes, with the clearing antibody/targeting protein-enzyme complex deposited into the liver where it is rapidly catabolized, i.e. inactivated. The anti-idiotypic antibody can optionally be substituted with carbohydrate/sugar residues such that the ensuing clearing antibody/targeting protein-enzyme complex is almost immediately sequestered by hepatic asialoglycoprotein receptors. In this method, no clearing antibody remains in circulation.

Alternatively, the anti-idiotypic monoclonal antibody is not glycosylated and hepatic localization of the antibody/targeting protein-enzyme complex takes place due to the large size of the complexes formed between the two antibodies in circulation. In this latter approach, uncomplexed clearing antibody remains in circulation for an extended period, with a similar clearance profile to any normal IgG, and will act to scavenge any targeting protein-enzyme conjugate which is released by the target site.

Although a clearing agent is used in accordance with one embodiment of the invention, the invention encompasses methods where no clearing agent is used. See, e.g., the examples set forth below.

Prodrugs

As used herein, the term “prodrug” means any compound that is converted to an active therapeutic agent (drug) by the enzyme of the targeting protein-enzyme conjugate. Useful therapeutic agents include toxins, antibiotic or chemotherapeutic drugs, radioisotopes, paramagnetic ions, boron addends, cytokines, photosensitizers, radiosensitizers, vasodilators and the like.

As discussed above, in accordance with one aspect of the present invention, the prodrug is a soluble substrate-therapeutic agent conjugate which, upon action by the enzyme, releases an insoluble therapeutic agent that has a substantially more favorable partition coefficient for attraction to the target site than the substrate-therapeutic agent conjugate.

As used herein, the term “soluble” means soluble in the fluid into which it is administered and by which it is transported to the target site, to a sufficient extent to permit transport of a therapeutically effective amount of the conjugate to the target site. Normally, administration will be into the bloodstream, by intravenous or intraarterial infusion, and the prodrug will be soluble in serum and preferably sufficiently hydrophilic to be carried largely by the aqueous phase of serum and diffuse relatively easily through the walls of the blood vessels into interstitial fluid, for cases where such is necessary. Where the target site is in the circulatory system, such as in cardiac therapy or therapy of atherosclerotic plaques, or the like, hydrophilic/lipophilic solubility is not as important.

In accordance with this embodiment, as the soluble substrate-therapeutic agent conjugate comes into contact with pretargeted enzyme, the soluble substrate-therapeutic agent conjugate releases an insoluble therapeutic agent that partitions favorably to the target. This partitioning out of the therapeutic agent after being acted upon by the targeted enzyme permits the drug to selectively accrete at the target site.

As used herein, the term “insoluble therapeutic agent” indicates a therapeutic agent that is less soluble than the substrate-therapeutic agent conjugate, and that partitions favorably to the target site, and includes therapeutic agents that are partially soluble, as long as they are less soluble than the substrate-therapeutic agent conjugate.

Certain cytotoxic drugs that are useful for cancer therapy are relatively insoluble in serum. Some that are quite toxic in unconjugated form have their toxicity considerably reduced by conversion to prodrug conjugates. Conversion of a relatively poorly soluble drug to a more soluble conjugate (prodrug), e.g., a glucuronide, will improve its solubility in the aqueous phase of serum and its ability to pass through venous, arterial or capillary cell walls and reach the interstitial fluid bathing the tumor. In fact, conversion of certain toxic substances such as aromatic or alicyclic alcohols, thiols, phenols and amines to glucuronides in the liver is the body's method of detoxifying them and making them more easily excreted in the urine.

In accordance with one embodiment of the invention, the prodrug is a low molecular weight compound, have a molecular weight of below about 50 KDa.

The general method of preparing a substrate-therapeutic agent conjugate (prodrug) according to the invention involves covalently binding at least one molecule or ion of therapeutic agent to a substrate for the enzyme of the targeting protein-enzyme conjugate. As mentioned above, one example of a soluble substrate-therapeutic agent conjugate is a glucuronide. The therapeutic agent may be attached to glucuronic acid to form the glucuronide, which solubilizes the conjugate. The attachment is usually to a hydroxyl, thiol or amine function of the therapeutic agent, which forms an acetal, thioacetal or aminoacetal with the aldehyde carbon of the glucuronic acid. This conjugate is cleaved by the enzyme glucuronidase, which would be the enzyme component of the targeting protein-enzyme conjugate. The released therapeutic agent is less soluble in the interstitial fluid than the glucuronide, and would tend to deposit on the cell membrane of surrounding cells and exert its cytotoxic effect at the site of localization of the targeting protein-enzyme conjugate.

One method of preparing such glucuronides is to inject a mammal, e.g., a cow, goat, horse or primate, with a cytotoxic drug. The drug is preferably administered by slow I.V. infusion, via a liver pump, through the hepatic artery or the portal vein. Some of the drug is converted to glucuronides in the liver of the animal, and the drug-glucuronide conjugate is then excreted in the urine. Collection of the urine and extraction of the glucuronide conjugate can then be effected, e.g., by ion exchange chromatography.

An alternative approach is to react UDP-glucuronic acid with the drug and then isolate the glucuronide from the reaction mixture. The reaction can be catalyzed by enzymes isolated from the endoplasmic reticulum of the liver of mammals, and/or the reaction can be carried out in the presence of extracts or tissue homogenates of the endoplasmic reticulum.

One type of antitumor drug that can be used in such a substrate-therapeutic agent conjugate is epirubicin, a 4′-epimer of doxorubicin (Adriamycin), which is an anthracycline glycoside and has been shown to be a substrate for human beta-D-glucuronidase. See Arcamone, Cancer Res., 45:5995 (1985). Other analogues with fewer polar groups would be expected to be more lipophilic and show greater promise for such an approach. Other drugs, toxins, boron compounds or chelators with aromatic or alicyclic alcohol, thiol or amine groups also would be candidates for such conjugate formation.

Another type of soluble substrate-therapeutic agent conjugate is a polymer with a plurality of agents linked thereto at intervals along the polymer backbone. The polymer can be one that is a substrate for the enzyme component of the targeting protein-enzyme conjugate or it can have segments or branches that are substrates for that enzyme. The therapeutic agent molecules are bound to the polymer in such a way that cleavage by the enzyme will liberate the therapeutic agent, rendering the therapeutic agent free of polymer units or bound to a small enough number of units to have the reduced solubility or favorable partition coefficient discussed above.

Examples of polymers for such use include polyols, polysaccharides, polypeptides and the like. One type of polysaccharide is dextran, an alpha-glycoside, which can be cleaved with the enzyme dextranase. The therapeutic agent can be functionalized to contain reactive groups towards the dextran hydroxyl groups, e.g., anhydrides, isocyanates or isothiocyanates and the like. Alternatively, dextran can be derivatized in a number of ways, e.g., by conversion to an amino-dextran.

One process for preparing a substrate-therapeutic agent conjugate with an aminodextran (AD) carrier starts with a dextran polymer, advantageously a dextran of average molecular weight (MW) of about 10,000-100,000, preferably about 10,000-40,000, and more preferably about 15,000. The dextran is then reacted with an oxidizing agent to effect a controlled oxidation of a portion of its carbohydrate rings to generate aldehyde groups. The oxidation is conveniently effected with glycolytic chemical reagents, e.g., NaIO₄, according to conventional procedures.

It is convenient to adjust the amount of oxidizing agent so that about 50-150, preferably 100 aldehyde groups are generated, for a dextran of MW of about 40,000, with about the same proportion of aldehyde groups for other MW dextrans. A larger number of aldehyde groups, and subsequent amine groups, is less advantageous because the polymer then behaves more like poylysine and may also be resistant to enzyme cleavage. A lower number results in less than desirable loading of therapeutic agent (i.e., drug, toxin, chelator, or boron addend), which may be disadvantageous, especially if the turnover number of the enzyme is low.

The oxidized dextran is then reacted with a polyamine, preferably a diamine, and more preferably a mono- or poly-hydroxy diamine. Suitable amines include, e.g., ethylene diamine, propylene diamine or similar polymethylene diamines, diethylene triamine or like polyamines, 1,3-di- amino-2-hydroxypropane or other like hydroxylated diamines or polyamines, and the like. An excess of the amine relative to the aldehyde groups can be used, to insure substantially complete conversion of the aldehyde functions to Schiff base (imine) groups.

Reductive stabilization of the resultant intermediate is effected by reacting the Schiff base intermediate with a reducing agent, e.g., NaBH₄, NaBH₃CN, or the like. An excess of the reducing agent is used to assure substantially complete reduction of the imine groups to secondary amine groups, and reduction of any unreacted aldehyde groups to hydroxyl groups. The resulting adduct can be further purified by passage through a conventional sizing column to remove cross-linked dextrans. An estimate of the number of available primary amino groups on the AD can be effected by reaction of a weighed sample with trinitrobenzenesulfonic acid and correlation of the optical density at 420 nm with a standard. This method normally results in essentially complete conversion of the calculated number of aldehyde groups to primary amine groups on the AD.

Alternatively, the dextran can be derivatized by conventional methods for introducing amine functions, e.g., by reaction with cyanogen bromide, followed by reaction with a diamine.

The AD is advantageously reacted with a derivative of the particular therapeutic agent (i.e, drug, toxin, chelator or boron addend), in an activated form, preferably a carboxyl-activated derivative, prepared by conventional means, e.g., using dicyclohexylcarbodiimide (DCC) or a water soluble variant thereof.

Methotrexate (MTX) is a typical drug for use in preparing conjugates according to the present invention. Activation of MTX is conveniently effected with any of the conventional carboxyl-activating reagents, e.g., DCC, optionally followed by reaction with N-hydroxysuccinimide (HOSu) to form the active ester. The reaction is normally effected in a polar, aprotic solvent, e.g., dimethylformamide (DMF), dimethylsulfoxide (DMSO) or the like. Other activated esters, e.g., p-nitrobenzoate and the like, can also be used, as can mixed anhydrides. The DCC/HOSu activation is mild and the activated MTX can be reacted in aqueous medium with the AD, so it is preferred.

The proportions of activated MTX to AD are preferably such that about half of the amino groups available on the AD react to form amide bonds with the carboxyl of the activated MTX. Thus, if about 100 amine groups are available on an AD with a starting MW of about 40,000, up to about 50 of these may be reacted with activated MTX. Using a proportion of about 50:1 MTX:AD, about 25-50 MTX molecules are normally introduced. It is difficult to achieve higher loading because of incipient precipitation of the adduct due to the increasing insolubility thereof.

Analogous steps can be used for other therapeutic agents, modified in appropriate ways which will be readily apparent to the ordinary skilled artisan. As an illustration of the adaptations to be used for other drugs, loading with 5-flourouracil (5-FU) can be effected by oxidizing 5-flourouridine at the carbohydrate portion, e.g., using periodate, reacting this intermediate with an aminodextran, and reductively stabilizing the Schiff base adduct. Cycloheximide can be loaded by direct reaction of its cyclohexanone carbonyl with aminodextran amine groups, followed by reductive stabilization, or by reacting its side chain hydroxyl with an excess of a diisothiocyanate linker and reaction of the isothiocyanate derivative with amines on the aminodextran, or by reaction of the imide nitrogen with e.g., a haloacid or haloester, followed by activation of the resultant carboxyl derivative, e.g., with DCC, and condensation with amines on the aminodextran.

Another illustration is provided by the antibiotic mitomycin C and its analogs. This molecule has an amine function and a cyclic imine, either of which can be reacted with an alkylating activating group, e.g., succinimidyloxy iodoacetate or sulfosuccinimidyloxy (4-iodoacetyl) aminobezoate (sulfo-SIAB), the resulting intermediate is then used to alkylate amine groups on an aminodextran. Alternatively, carboxyl groups can be introduced using, e.g., succinic anhydride, then activated, e.g., with DCC, and the activated intermediate coupled as before.

Toxins, e.g., pokeweed antiviral protein (PAP) or the ricin A-chain, and the like, can be coupled to aminodextrans by glutaraldehyde condensation or by reaction of activated carboxyl groups on the protein with amines on the aminodextran.

Many drugs and toxins are known which have a cytotoxic effect on tumor cells or microorganisms that may infect a human and cause a lesion, in addition to the specific illustrations given above. They can be found in compendia of drugs and toxins, such as the Merck Index and the like. Any such drug can be loaded onto an AD by conventional means well known in the art, and illustrated by analogy to those described above.

Conjugates of therapeutic agents and PEG also are useful in the present invention. PEG generally has a solubilizing effect, and, accordingly, will enhance the difference in solubility between the administered PEG-therapeutic agent conjugate and the released therapeutic agent. In one aspect of this embodiment, a PEG molecule is conjugated at both its free hydroxyl ends to a therapeutic agent moiety. Such an agent will be long-circulating, giving the targeted targeting protein-enzyme conjugate ample time to act on the PEG-therapeutic agent conjugate to release active therapeutic agent. In one specific aspect of this embodiment, the therapeutic agent is attached to the PEG molecule through a linker. In this embodiment, the linker can be selected such that it is acted on by the enzyme of the targeting protein-enzyme conjugate to release the therapeutic agent. For example, a peptide linker that is cleaved by cathepsins can be used in this and other embodiments of the invention.

The ability to partially or completely detoxify a therapeutic agent as a conjugate while it is in circulation can reduce systemic side effects of the drug and permit its use when systemic administration of the unconjugated drug would be unacceptable. For example, MTX and cycloheximide often are too toxic when administered systemically. Administration of more molecules of the drug conjugated to a substrate carrier, according to the present invention, permits therapy while mitigating systemic toxicity.

An alternative to an alpha-glycoside such as dextran or aminodextran is a beta-glycoside such as carboxymethylcellulose (CMC), which can be cleaved by a cellulase enzyme. Attachment of therapeutic agents to the CMC is analogous to the method used for dextran, since both are sugar polymers, differing only in the stereochemistry of the glycosidic linkage. Derivatization of the CMC to append functional molecules is perhaps most conveniently accomplished by reacting it with a carbodiimide type of condensing agent and using an amine function on the diagnostic or therapeutic agent to form amide linkages. Alternatively, mild oxidation with glycol cleavage reagents, e.g., periodate, to form aldehyde groups at a plurality of points along the polymer chain, followed by reaction with a diamine, will form an aminoCMC suitable for reaction with a variety of different functional groups. Condensation of the oxidized CMC with amines and borohydride stabilization is also practicable. Other means of attachment of agents to the CMC will be readily apparent to the ordinary skilled artisan.

Yet another variant on the polymer substrate is a polymer that is not cleaved by the enzyme, but which bears short linker segments of an oligomer that is a substrate for the enzyme, and which bears drugs, chelators, boron addends and like therapeutic agents. As one illustration, a polyvinyl alcohol could be used as a carrier for a plurality of short oligosaccharides, e.g., short dextran or cellulose oligomers of, e.g., 5-50, preferably 5-20, glycoside subunits. The polyvinyl alcohol could be aminated by, e.g., cyanogen bromide followed by diamine condensation. The oligosaccharide could be mildly oxidized with, e.g., periodate, and condensed with the aminated polymer to form Schiff base linkages, which are preferably further stabilized by borohydride reduction. The resultant oligomer-charged polymer can then be lightly aminated as described earlier for dextran or cellulose polymers, or otherwise conventionally functionalized, to put at least 2, preferably about 2-5, amine groups on each of the oligomer linkers. An average of about 1-3 drug molecules, chelators, boron addends or other agents is then conjugated to each of the oligomers.

Loading of drugs on the substrate carrier will depend upon the solubility (partition coefficient between the fluid bathing a target site and the target cells, tissues or other structures, e.g., atherosclerotic plaque, fibrin clot, virus particle, parasite and the like), and upon the efficiency of enzyme cleavage of the substrate molecules or subunits to liberate the therapeutic agent, which has a sufficiently favorable partition coefficient to the target to effect the desired therapeutic action. In general, it will be desirable to load, for example, a drug onto a dextran in a ratio of monosaccharide subunits to drug of from about 3 to about 25, although these are preferred and not limiting amounts. Very heavy loading of drug molecules can inhibit enzyme activity due primarily to steric hindrance to binding of the substrate conjugate to the active site of the enzyme. Too light loading can result in insufficient reduction in fluid solubility for the drug as a result of enzyme cleavage since a smaller portion of the polysaccharide-drug conjugate might diffuse away from the bound enzyme before enough sugar subunits are cleaved off to reduce solubility enough for the drug (with perhaps a few glycoside subunits still bound to it) to be favorably partitioned out of the surrounding fluid to accrete at the target site, e.g., on a tumor cell membrane, on a bacterial cell wall, on an atherosclerotic plaque or a fibrin clot and the like. Toxins may be less heavily loaded than other drugs, since they are often larger proteins.

Chelators for radiometals or magnetic resonance enhancers are well known in the art. Typical are derivatives of ethylenediamine-tetraacetic acid (EDTA) and diethylenetriaminepentaacetic acid (DPTA). These typically have groups on the side chain by which the chelator can be attached to a carrier. This same group can be used to couple the chelator to amine groups on an AD. Alternatively, carboxyl or amine groups on a chelator can be coupled to an AD activation or prior derivatization and then coupling, all by well known methods. For example, deferoxamine, which is a chelator for Ga-67, has a free amine group that can be activated with a suitable linker to contain an activated carboxyl, isothiocyanate or like group, and then coupled to amines on an AD. Other methods of linking chelators to amines of an AD will be apparent to the skilled artisan, depending on the functionality of the chelator. As discussed above, betaglycosides can be used instead of AD by methods known in the art.

It will be readily apparent that many other variants can be envisioned. The condensation of the lightly oxidized dextran oligomer linkers to the aminated polymer and the drug or other agent can be effected simultaneously or sequentially and stabilized later. Other functional groups on the agents can be used to bind to the oligomer, and other functional groups can be used to bind the oligomer to the polymer carrier.

An acrylate polymer can be used, with aminodextran oligomers bound to it by amide linkages formed by carbodiimide activation of the acrylate carboxyls. A polypeptide can be used, with the oligomer linkers attached to carboxyl or amine residues on the carrier. A short polyester or oligopeptide linker can be used instead of an oligosaccharide linker, with an esterase or peptidase enzyme that cleaves the linker. The ordinary skilled artisan will be able to envision other variants that fall within the broad scope of the invention and can be prepared by conventional synthetic methods.

Still another approach is to use a carrier polymer that bears the drugs, chelators, boron addends or other therapeutic agents and that has a high attraction for the target, in unmodified form, but which is then modified by conjugation to solubilizing substrate molecules which are then cleaved by targeted enzyme. One illustration of this type of substrate-therapeutic agent conjugate is a polylysine to which are bound a plurality of radiometal or paramagnetic metal chelators, carboranes or MTX molecules. This carrier conjugate is then condensed with a plurality of short dextran oligomers, e.g., by Schiff base formation with lightly oxidized dextran and borohydride stabilization, in a ratio which increases the solubility (reduces the “stickiness”) of the polylysine and makes it readily trans-portable in serum and readily diffusable through capillary walls (and then loaded with radioisotope or paramagnetic ions, if chelators are attached to the carrier). At a target site, e.g., a tumor, a localized antitumor antibody-dextranase conjugate would strip off the dextran coating from the polylysine to a sufficient extent to make it “sticky” again, whereupon it would adhere to the tumor cells and or therapeutic agent would then act upon the tumor cell to permit imaging or cytotoxic therapy.

A heavily aminated aminodextran can function as a polylysine, and can be substituted with short oligomeric substrate linkers as discussed above. It will be “sticky” towards cell membranes and other tissues in a similar fashion to polylysine. Other polypeptides or heavily aminated polymers can function in analogous fashion as carriers for substrate coating and solubilization. In fact, amination is not essential to the loaded carrier function, since any functionality that causes favorable partitioning out of a conjugate of a carrier and one or more diagnostic and/or therapeutic agents can be masked by solubilizing substrate oligomers or even small substrate molecules, such that the resultant more soluble conjugate circulates easily in serum or another fluid for administration and becomes less soluble in the fluid bathing the target site after the coating molecules are cleaved by action of the localized enzyme of the targeting protein-enzyme conjugate.

The proportions of loaded carrier polymer to “coating” solubilizing substrate groups or oligomers will depend on the nature of the target site and the characteristics of the components. If a polylysine or functional equivalent is used as the carrier, coating with oligodextran will advantageously be effected in a dextran:polylysine ratio of about 1:10 to about 100:1 by weight, preferably about 1:1 to about 10:1, more preferably about 3:1 to about 7:1. An example is a polylysine of MW about 1,500 daltons, coated with about 3-7 dextran oligomers of MW about 15,000 daltons each.

In one embodiment, a therapeutic method of the invention is accomplished by conjugating an effective therapeutic amount of a radioisotope such as Y-90 or I-131 (which may be used for both localization and therapy depending on the amount injected) or a drug such as adriamycin for cancer or gentamycin for infection, an immunomodulatory substance such as poly-IC, or a biological toxin such as pokeweed mitogen to the enzyme substrate, and depositing a therapeutically effective amount of the agent at the target site.

Boron addends, e.g., carboranes, when attached to the substrate conjugate and targeted by the targeting protein to target sites, can be activated by thermal neutron irradiation and converted to radioactive atoms which decay by alpha emission to produce highly cytotoxic short-range effects. High loading of boron addends, as well as magnetic resonance enhancing ions, is of great importance in potentiating their effects. Carboranes can be made with carboxyl functions on pendant side chains, as is well known in the art. Attachment of these carboranes to AD's by activation of the carboxyl groups and condensation with amines on the carriers enables preparation of useful substrate-agent conjugates. Carboranes linked to other polymers, such as PEG discussed above, also are useful in this embodiment. Particularly useful are carboranes linked to a polymer via a peptide that is cleaved by the enzyme of the targeting protein-enzyme conjugate to release the carborane-boron compound.

In one embodiment of the present invention, the substrate-therapeutic agent conjugate contains a large number of boron atoms, preferably prepared from reagents enriched in Boron-10 isotope, boron containing reagents enriched to about 96% Boron-10 being commercially available. Such a conjugate would be of great utility in neutron activated radiotherapy, since it could bring to a tumor site or the site of a pathological lesion a sufficient number of boron atoms to provide a therapeutic dosage of alpha particles to the targeted tissue upon thermal neutron irradiation, even when the percentage of an injected dose of targeting protein-enzyme conjugate which localizes in the target tissue is relatively low, e.g., 1-10%.

The boron-loaded prodrugs according to the present invention have a number of boron atoms per substrate molecule normally ranging from at least about 50 up to about 10,000, preferably from about 200 to about 2,000. To reiterate, these are preferably about 96% Boron-10 enriched, although it may be more cost effective to use a conjugate having a larger number of boron atoms with the 20% natural abundance of Boron-10 isotope.

The prodrugs can contain moieties which do not contain boron, or which contain boron and other useful functions, e.g., a radionuclide, especially I-123, I-125 or I-131, or functions such as chelators, chelates with metal ions, drugs, toxins, chromophores, chromogens, fluorescent markers, and the like, all of which can contribute to the therapeutic effect thereof, or permit monitoring of deposit and/or clearance of the boron addend, or provide complementary therapeutic activity. The substrate-agent conjugate may incorporate functions whose primary purpose is to improve the lipid solubility and decrease the water solubility of the resultant enzyme cleavage product containing the boron addend.

It is useful to employ boron cage compounds to make such conjugates, because of their ability to carry 5-12 boron atoms to the target site. The most common and readily available kinds of boron cage compounds are the carboranes. The skilled artisan will be aware of general references in the field for most of the reactions to be discussed hereinafter, the best and most comprehensive references being Muetterties et al., “Polyhedral Boranes”, (Dekker, New York, 1968); Muetterties, Ed., “Boron Hydride Chemistry”, (Academic Press, New York, 1975); and Grimes, “Carboranes”, (Academic Press, New York, 1970). These references contain copious bibliographies on specific topics within the broad subject range of synthesis of organic derivatives containing a plurality of boron atoms. Hawthorne, U.S. patent application Ser. No. 742,436, filed Jun. 7, 1985, is replete with such details, and this application is incorporated in its entirety herein by reference.

Thus, in one embodiment, a therapeutic method of the invention is accomplished by conjugating one or more boron-10 addends to a substrate for the enzyme and, once the boron-10 is deposited at the target site, effecting external thermal neutron irradiation to the tumor to destroy the neoplastic cells. In accordance with one embodiment of the invention, the boron-10 conjugate is labeled with a detectable label, such as a radioisotope chelate, to permit the practitioner to ensure that sufficient boron addends have localized at the target site and that substantially all of the non-targeted boron-10 has cleared the circulatory system prior to neutron irradiation.

Routes of Administration and Dosages

The method of the invention is normally practiced by parenterally injecting compositions comprising the targeting protein-enzyme conjugate, the (optional) clearing agent, and the prodrug. Suitable types of parenteral injections include intracavitary (e.g., intraperitoneal), intravenous, intraarterial, intrapleural, intrathecal, intramuscular, intralymphatic and regional intraarterial, intralesional, subcutaneous, catheter perfusion and the like. It will be appreciated that the modes of administration of the different compositions may not be the same, since the clearance pathways and biodistributions of the conjugates will generally differ.

For cancer therapy, intravenous, intraarterial or intrapleural administration is normally used for lung, breast, and leukemic tumors. Intraperitoneal administration is advantageous for ovarian tumors. Intrathecal administration is advantageous for brain tumors and leukemia. Subcutaneous administration is advantageous for Hodgkin's disease, lymphoma and breast carcinoma. Catheter perfusion is useful for metastatic lung, breast or germ cell carcinomas of the liver. Intralesional administration is useful for lung and breast lesions.

The targeting protein-enzyme conjugate may be administered as an aqueous solution in PBS, preferably as a sterile solution, especially if it is for use in humans. Advantageously, dosage units of about 50 micrograms to about 5 mg of the targeting protein-enzyme conjugate will be administered, either in a single dose or in divided doses, although smaller or larger doses may be indicated in particular cases. It may be necessary to reduce the dosage and/or use targeting proteins from other species and/or hypoallergenic antibodies, e.g., fragments or hybrid human or primate antibodies, to reduce patient sensitivity, especially for therapy and especially if repeated administrations are indicated for a therapy course or for additional diagnostic procedures. An indication of the need for such cautionary procedures is an increase in human anti-mouse antibody (HAMA) production, which can be determined using an immunoassay.

It may take from about 2 to 14 days and frequently takes from 5 to 14 days for IgG antibody to localize at the target site and substantially clear from the circulatory system. The corresponding localization and clearance time for F(ab)₂ and F(ab′)₂ antibody fragments is from about 2 to 7 days and frequently from 4 to 7 days, and from about 1 to 3 days and frequently from 3 days for Fab and Fab′ antibody fragments. Other antibodies may require different time frames to localize at the target site, and the above time frames may be affected by the conjugated enzyme. As discussed above, by labeling the targeting protein-enzyme conjugate, its localization and clearance can be monitored. As discussed above, clearance time can be minimized by the use of a clearing agent.

IgG is normally metabolized in the liver and, to a lesser extent, in the digestive system. F(ab)₂ and F(ab′)₂ are normally metabolized primarily in the kidney, but can also be metabolized in the liver and the digestive system. Fab and Fab′ are normally metabolized primarily in the kidney, but can also be metabolized in the liver and the digestive system.

Preferably, at least about 0.0001% of the injected dose of targeting protein-enzyme conjugate is permitted to localize at the target site before the prodrug is administered. To the extent that a higher targeting efficiency for this conjugate is achieved, this percentage can be greater, and a reduced dosage can be administered.

It follows that an effective amount of an targeting protein-enzyme conjugate is that amount sufficient to target the conjugate to the antigen at the target site and thereby bind an amount of the enzyme sufficient to convert enough of the prodrug to active therapeutic agent to achieve accretion of a therapeutically effective amount of the therapeutic agent at the target site.

The prodrug also may be administered as an aqueous solution in PBS. Again, this should be a sterile solution if intended for human use. As described above, the prodrug is administered after a sufficient time has passed for the targeting protein-enzyme conjugate to localize at the target site and substantially clear from the circulatory system of the patient.

An effective amount of prodrug is that amount sufficient to release an effective amount of the therapeutic agent at the target site and advantageously is that amount which will be capable of conversion by the enzyme to a form of the therapeutic agent that tends to accumulate at the target site. An effective amount of a therapeutic agent is that amount sufficient to treat the target site.

Dosage units of prodrug will depend upon many factors, each of which can be determined by the skilled artisan in a straightforward manner, so that optimal dosimetry can be effected. It may be helpful, in the initial dosimetric evaluation, to use a radiolabeled prodrug (if the therapeutic agent is not itself a radioactive isotope) to determine the degree and rate of deposit of the therapeutic agent at the target site, and the rate of clearance and biodistribution of non-targeted prodrug. The radiolabelling of prodrug can be effected in much the same way as the radiolabelling of the targeting protein-enzyme conjugate discussed above. Use of a labeled targeting protein-enzyme conjugate to estimate the amount of enzyme localized at the target site also will aid in dosimetric analysis.

It is possible to perform trials for dosimetry, generally using an animal model first, if available, then a series of patient studies, to optimize the dose of prodrug as a function of accessibility of the target site, mode of administration, turnover number of the enzyme, desired dose of the therapeutic agent at the site, and rate of optimization is expected in the art, and techniques for optimization are well-within the ordinary skill of the clinician.

Kits

The present invention also provides compositions and kits for carrying out the above-described methods. One embodiment of the invention provides injectable preparations for human therapeutic use. A first injectable preparation comprises an effective amount for targeting and converting prodrug to therapeutic agent of at least one targeting protein conjugated to at least one enzyme in a pharmaceutically acceptable injection vehicle, such as phosphate-buffered saline (PBS) at physiological pH and concentration. A second injectable preparation comprises an amount effect for therapy of a prodrug in a pharmaceutically acceptable injection vehicle, such as that used for the first preparation. Optionally, another injectable preparation comprising a clearing agent is provided. Alternatively, if the targeting protein-enzyme conjugate is to be formed in situ, two injectable preparations are provided in place of the first preparation described above, one comprising the targeting protein, and one comprising the enzyme. The injectable preparations preferably are sterile, particularly if they are intended for use in humans.

Another embodiment of the invention provides a kit for effecting therapy in accordance with the present invention. A first container comprises an amount effective for targeting and converting prodrug of at least one targeting protein conjugated to at least one enzyme. A second container comprises an amount effective for therapy of a prodrug. Optionally, a third container is provided comprising a clearing agent. Alternatively, if the targeting protein-enzyme conjugate is to be formed in situ, two containers are provided in place of the first contained described above, one comprising the targeting protein, and one comprising the enzyme. The compositions can be lyophilized for longer shelf stability or provided in the form of solutions, optionally containing conventional preservatives, stabilizers and the like. Other optional components of such kits include buffers, labeling reagents, radioisotopes, paramagnetic compounds, conventional syringes, columns, vials and the like.

Without further elaboration, it is believed that one skilled in the art using the preceding description can utilize the present invention to its fullest extent. The following examples are illustrative only, and are not intended to limit the scope of the invention. In the following examples, all temperatures are set forth uncorrected in degrees Celsius; unless otherwise indicated, all parts and percentages are by weight.

### EXAMPLES

I. Targeting Proteins

Preparation of Bispecific Antibody Specific for Two Different Tumor-Associated Antigens:

The interchain disulfide bridges of an F(ab′)₂ fragment having specificity for a tumor-associated antigen (i.e., carcinoembryonic antigen (CEA)) is gently reduced with cysteine, taking care to avoid light-heavy chain linkage, and the SH group(s) are activated with an excess of bis-maleimide linker (1,6-bis(maleimido)hexane) to form Fab′-maleimide fragments. An antibody specific for a different tumor-associated antigen (i.e., colon-specific antigen p) is converted to Fab′-SH and then reacted with the first Fab′-SH fragment to obtain a bispecific antibody.

Preparation of Bispecific Antibody Specific for a Tumor-Associated Antigen and an Enzyme:

The interchain disulfide bridges of an F(ab′)₂ fragment having specificity for a tumor-associated antigen (i.e., CEA) is gently reduced with cysteine, taking care to avoid light-heavy chain linkage, to form Fab′-SH fragments. The SH group(s) are activated with an excess of bis-maleimide linker (1,6-bis(maleimido)hexane). An antibody specific for an enzyme is converted to Fab′-SH and then reacted with the first Fab′-SH fragment to obtain a bispecific antibody, with specificity for the target site and the enzyme.

II. Targeting Protein-Enzyme Conjugates

Preparation of Antibody-Enzyme Conjugate

(a) A substantially monoconjugated enzyme-antibody preparation is prepared by mildly oxidizing the carbohydrate portion of an anti-CEA IgG with periodate, then contacting the oxidized IgG with a dilute solution of dextranase (from Penicillium sp., Worthington Biochemical Corp., Freehold, N.J.) to produce an antibody-enzyme conjugate, which is then stabilized by borohydride, in the usual manner. The conjugate can be radiolabeled with I-131, by conventional procedures.

(b) In a similar fashion to the above Part A, an anti-leukemia IgG is conjugated to glucuronidase (from bovine liver, Worthingon).

Preparation of Antibody-Enzyme Conjugate Using a Bi-Specific Antibody

(a) A bispecific antibody having specificity for a tumor-associated antigen and for an enzyme is contacted with the enzyme, and antibody-enzyme conjugate is isolated from non-conjugate antibody and enzyme by conventional means.

(b) Alternatively, a bispecific antibody having specificity for a tumor-associated antigen and for an enzyme is made in situ by administering the antibody, permitting the antibody to localize at the target site and substantially clear from circulation, and then administering the enzyme before administering the enzyme.

III. Prodrugs

Preparation of Epirubicin-Glucuronide Conjugate

Epirubicin is injected intravenously into a horse over a period of several weeks. Urine is collected, and epirubicin glucuronide is isolated by ion-exchange chromatography of the urine, and purified by further column chromatography and/or HPLC.

Preparation of Methotrexate/Aminodextran Conjugate

(a) Activation of Methotrexate

In a dried Reacti-vial, 45.4 mg of methotrexate (0.1 mmole, Sigma) in 1 ml anhydrous DMF is introduced by syringe. A solution of N-hydroxy-succinimide (23 mg, 0.2 mmole, Sigma) in 7590 μl of anhydrous DMF and a solution of 1,3-dicyclohexylcarbodiimide (41.5 mg, 0.2 mmole, Sigma) in 750 μl of anhydrous DMF are followed. The reaction mixture is stirred in the dark at room temperature for 16 hours under anhydrous conditions. The white precipitate is centrifuged and the clear solution is stored in a sealed bottle at −20° C.

(b) Reaction with Aminodextran

Aminodextran (10 mg, 2.5×10⁴ mole) is dissolved in 2 ml of PBS, pH 7.2. Activated MTX (125×10⁴ mmole) is added gradually. The solution is stirred at room temperature for 5 hours and purified by Sephadex G-25 column. The void volume is collected and further dialyzed against reaction buffer. After lyophilization, 2.1 mg of product is obtained (21% yield). The methotrexate incorporation is determined by the absorption at 370 nm to be 38 methotrexate/dextran.

IV. Clearing Agents

Galactosylation of the Anti-Idiotypic WI2 Antibody

The WI2 antibody is described in Losman et al., Int. J. Cancer 56:580-584 (1994), the contents of which are incorporated by reference. It is an anti-idiotypic antibody to the murine anti-CEA mAb, MN-14.

Proteins are glycosylated according to the method outlined by Ong et al., Cancer Res. 51: 1619-1626, (1991). Briefly, cyanomethyl-2,3,4,6-tetra-O-acetyl-1-thio-β-D-galactopyranoside (Sigma Chemical Co., St. Louis Mo.) is dissolved in dry methanol to give a 0.1 M solution and then mixed with a 1/10th volume of sodium methoxide (J. T. Baker Chemical Co., Phillipsburg N.J.). The flask contents are stoppered to prevent absorption of water, and then left to stand 48 hours at room temperature. The solution may be stored for a period of months in the refrigerator.

For conjugation to each mg of WI2 antibody, 500 μl of the imido ester solution is evaporated to dryness and then redissolved in a 1 mg/ml solution of WI2 in 0.25M sodium borate buffer, pH 8.5. After standing for 2 hours, the galactosylated WI2 is dialyzed into 0.1M phosphate buffered saline, pH 7.4.

Effect of Galactose Substitution on WI2

WI2 was treated with excess thioimidate to introduce various levels of galactose (11, 22, 48 and 76% lysines modified). To determine the effect of galactose substitution on blood clearance, these proteins were radiolabelled with I-131 and injected into BALB/c mice. Animals were bled after 1 h and sacrificed 24 h post-injection. The amount present in blood at 1 h is shown in Table 3.

TABLE 3
% ID¹³¹I-galₓ WI2/g blood at 1 hour

| % Modified lysines | % ID/g |
|---|---|
| 0 | 45.96 ± 5.203 |
| 11 | 51.19 ± 1.443 |
| 22 | 42.57 ± 2.545 |
| 48 | 5.92 ± 0.704 |
| ≧76 | 0.7 ± 0.2 |

TABLE 2
% ID¹³¹I-galₓ WI2/g after 24 hours

% ID/g

| % Modified lysines | Blood | Liver |
|---|---|---|
| 0 | 22.81 ± 3.101 | 6.49 ± 1.394 |
| 11 | 23.31 ± 1.185 | 6.98 ± 0.798 |
| 22 | 17.04 ± 1.561 | 7.66 ± 0.752 |
| 48 | 3.26 ± 0.694 | 33.19 ± 2.335 |
| ≧76 | 0.2 ± 0.006 | 19.2 ± 7.0 |

V. Methods

Therapy of Lung Cancer

A human patient having small-cell carcinoma of the right lung is infused intravenously with a sterile, pyrogen-free solution containing 5 mg of an anti-CEA IgG/dextranase conjugate in PBS, prepared according to the above example and labeled with I-131. After 5 days, the conjugate is well localized in the lung and has substantially cleared from the circulation of the patient, as seen by scintigraphic scanning at daily intervals.

A sterile, pyrogen-free PBS solution of a MTX/aminodextran conjugate prepared according to the above example and containing 50 mg of the conjugate, is infused intravenously on each of the next 4 days. Subsequent radioimmunodetection with I-123-anti-CEA Fab shows significant tumor reduction.

Therapy of Lymphoma

A human patient suffering from lymphoma is infused intravenously with a sterile, pyrogen-free PBS solution containing 5 mg of an anti-lymphoma IgG-glucuronidase conjugate prepared according to the example above, and labeled with I-131. After 6 days, the conjugate is well localized at the target site and substantially cleared from the circulatory system, as determined by gamma scanning.

The patient is then infused intravenously with a sterile, pyrogen-free PBS solution containing 10 mg of epirubicin glucuronide, prepared according to the example above, on each of the next 4 days. Subsequent radioimmunodetection shows significant reduction in the lymphoma.

Tumor Therapy With Anti-Idiotypic Clearing Step

A patient having a tumor expressing carcinoembryonic antigen (CEA) is treated with an antibody conjugate with at least one arm which is reactive with CEA. This specificity is exemplified by the MN-14 antibody. The antibody is linked to the enzyme carboxyesterase. After tumor accretion of antibody conjugate has reached a maximum, a dose of an anti-idiotypic antibody to MN-14, termed WI2, is given in sufficient amount to clear non-targeted antibody conjugate from the circulation. Later, the patient is treated with the camptothecin prodrug CPT-11. The presence of MN-14-carboxyesterase at the tumor surface induces its hydrolysis in situ at the tumor to generate the highly toxic metabolite SN-38. The latter is much more insoluble than CPT-11 and being at least one hundred-fold more toxic, its anti-tumor effects are multiplied accordingly.

Tumor Therapy With Dual Therapeutic Agents and Anti-Idiotypic Clearing Step

A patient with a CEA-producing cancer is treated with Iodine-131-radiolabeled humanized IMMU-14-carboxypeptidase-G2 conjugate. At the time of maximum tumor accretion of the antibody-enzyme conjugate a sufficient does of the humanized anti-idiotypic antibody WI2 is given to clear circulating excess Iodine-131-radiolabeled humanized IMMU-14-carboxypeptidase-G21 conjugate. The patient is then treated with an injection of CPT-11, given either as a bolus injection or as a continuous infusion, either being given according to standard procedures for administration of this drug.

Cancer Therapy with Multiple Antibody-Enzyme Conjugates

A patient presenting with colo-rectal cancer is given an injection of IMMU-14-esterase G2 (anti-carcinoembryonic antigen mAb) and Mu-9-glucuronidase (anti-colon-specific antigen p mAb). After sufficient time for targeting and clearance to occur the patient is given an injection of the camptothecin prodrug CPT-11. That prodrug which localizes to the IMMU-14-esterase at the tumor is cleaved into the active metabolite SN-38. SN-38 which is not localized is mainly cleared after substantial conversion to its glucuronide form. In this instance the added presence of the Mu-9-glucuronidase conjugate also localized at the tumor site ensures that circulating SN-38-glucuronide is also cleaved at the tumor site and reactivated to SN-38.

The preceding examples can be repeated with similar success using other composition and reaction conditions described above in the Detailed Description.

Various changes and modifications of the invention can be made by those skilled in the art to adapt the invention to various usages and conditions without going beyond the scope of the invention.

What is claimed is:

1. A method for targeting a therapeutic agent to a target site in a patient, comprising the steps of:
(a) administering to the patient an effective amount for targeting of at least one multispecific targeting protein comprising at least one first binding site which specifically binds to a substance produced by or associated with the target site and present at the target site and at least one second binding site which specifically binds to an epitope on an enzyme, wherein binding between the targeting protein and the enzyme does not interfere with enzyme activity;
(b) optionally, administering to the patient an amount effective for clearance of a first clearing composition comprising a clearing agent which clears non-localized targeting protein from circulation;
(c) administering to the patient an effective amount for enzyme activity of the enzyme, such that the targeting protein binds the enzyme to form a targeting protein-enzyme conjugate in situ;
(d) optionally, administering to the patient an amount effective for clearance of a second clearing composition comprising a clearing agent which clears non-localized targeting protein, non-localized enzyme, or non-localized targeting protein-enzyme conjugate from circulation;
(e) administering to the patient at least one serum-soluble prodrug composition, wherein the enzyme administered in step (c) acts on the prodrug to release a therapeutic agent that is less soluble in serum than the prodrug, and wherein the therapeutic agent partitions out at the target site such that it accretes at the target site to a greater extent than would the prodrug, thereby providing therapeutic agent at the target site.

2. The method of claim 1, wherein the targeting protein comprises a conjugate of at least one antibody, antibody fragment, or antibody subfragment which provides the first binding site and at least one antibody, antibody fragment or antibody subfragment which provides the second binding site.

3. The method of claim 1, wherein the targeting protein comprises a fusion protein comprising the first and second binding sites.

4. The method of claim 1, wherein the targeting protein comprises a covalent conjugate of at least two single chain antibodies, wherein at least one single chain antibody provides the first binding site and at least one single chain antibody provides the second binding site.

5. The method of claim 1, wherein the enzyme is selected from the group consisting of esterases, proteases, glucuronidases, dextranases, cellulases, and glycosidases.

6. The method of claim 5, wherein the enzyme comprises carboxyesterase.

7. The method of claim 5, wherein the enzyme comprises an esterase and the prodrug comprises CPT-11.

8. The method of claim 5, wherein the enzyme comprises beta-glucuronidase and the prodrug comprises CPT-11.

9. The method of claim 5, wherein the targeting protein comprises a conjugate of hMN-14 and anti-human-beta-glucuronidase, and the enzyme comprises human-beta-glucuronidase.

10. The method of claim 5, wherein the second binding site of the targeting protein is specific for DTPA, and the enzyme comprises a DTPA-human-beta-glucuronidase conjugate.

11. The method of claim 5, wherein the enzyme is selected from the group consisting of dextranases and cellulases, and wherein the prodrug comprises an aminodextran or polylysine carrier that is not acted on by the enzyme, to which is conjugated at least one molecule or ion of therapeutic agent, and which is further conjugated to at least one solubilizing dextran or carboxymethylcellulose oligomer which is acted on by the enzyme.

12. The method of claim 1, wherein the enzyme is selected from the group consisting of alkaline phosphatase, beta-lactamase, and carboxypeptidase G2.

13. The method of claim 12, wherein the enzyme comprises carboxypeptidase G2 and the prodrug comprises CPT-11.

14. The method of claim 1, wherein the prodrug is a low molecular weight compound, with a molecular weight below about 50 KDa.

15. The method of claim 14, wherein the prodrug comprises a benzoic or phenolic or anilic derivative of mustard.

16. The method of claim 1, wherein the prodrug comprises a glucuronide conjugate of the therapeutic agent.

17. The method of claim 1, wherein the prodrug comprises a polymer.

18. The method of claim 17, wherein the polymer is a dextran, an aminodextran, a carboxymethylcellulose or a polypeptide.

19. The method of claim 17, wherein the prodrug comprises a polyethylene glycol (PEG) molecule conjugated at both its free hydroxyl ends to one molecule or ion of the therapeutic agent.

20. The method of claim 17, wherein the polymer is conjugated to the therapeutic agent via a peptide linker.

21. The method of claim 20, wherein the peptide linker is cleaved by a cathepsin, and wherein the enzyme comprises a cathepsin.

22. The method of claim 17, wherein the prodrug comprises a polymer that is not acted on by the enzyme to which is attached at least one oligomer to which is conjugated at least one molecule or ion of therapeutic agent, wherein the oligomer is acted on by the enzyme.

23. The method of claim 1, wherein the targeting, protein comprises at least two first binding sites which specifically bind to the same or different epitopes of the same or different substance produced by or associated with the target site and present at the target site.

24. The method of claim 1, wherein step (a) comprises administering at least two different targeting proteins each of which comprise a first binding site which specifically binds to a different substance produced by or associated with the target site and present at the target site.

25. The method of claim 1, wherein the targeting protein comprises at least two second binding sites which specifically bind to different enzymes, and wherein step (c) comprises administering the different enzymes.

26. The method of claim 1, wherein step (a) comprises administering at least two different targeting proteins each of which comprise a second binding site which specifically binds to a different enzyme, and wherein step (c) comprises administering the different enzymes.

27. The method of claim 26, wherein step (c) results in the in situ formation of a targeting protein-enzyme conjugate comprising carboxypeptidase G2 and a targeting protein-enzyme conjugate comprising glucuronidase.

28. The method according to claim 27, wherein the prodrug comprises CPT-11.

29. The method of claim 26, wherein step (c) results in the in situ formation of a targeting protein-enzyme conjugate comprising esterase and a targeting protein-enzyme conjugate comprising glucuronidase.

30. The method according to claim 29, wherein the prodrug comprises CPT-11.

31. The method of claim 1, wherein the targeting protein, the enzyme, or both, is labeled with a detectable label such that step (c) results in the in situ formation of a labeled targeting protein-enzyme conjugate, and the method further comprises, prior to step (e), detecting the detectable label to determine the location of the targeting protein-enzyme conjugate.

32. The method of claim 31, wherein the detectable label is selected from the group consisting of radioisotopes and magnetic resonance image enhancing agents.

33. The method of claim 1, wherein the targeting protein, the enzyme, or both, comprises a therapeutic agent such that step (c) results in the in situ formation of a targeting protein-enzyme conjugate comprising a therapeutic agent.

34. The method of claim 33, wherein the therapeutic agent of the targeting protein-enzyme conjugate is selected from the group consisting of radionuclides, drugs, toxins, and boron addends.

35. The method of claim 33, wherein the therapeutic agent of the targeting protein-enzyme conjugate is a radionuclide selected from the group consisting of I-131, I-125 and At-211.

36. The method of claim 1, wherein the optional clearing composition of step (b) is administered.

37. The method of claim 36, wherein the clearing agent is anti-idiotypic to a first binding site of the targeting protein.

38. The method of claim 37, wherein the clearing agent is an anti-idiotypic monoclonal antibody.

39. The method of claim 36, wherein the clearing agent is modified with a number of sugar residues sufficient to effect hepatic clearance substantially in a single pass.

40. The method of claim 39, wherein at least about 48% of the lysine residues of the clearing agent are modified with sugar residues.

41. The method of claim 40, wherein at least about 76% of the lysine residues of the clearing agent are modified with sugar residues.

42. The method of claim 1, wherein the therapeutic agent comprises a boron addend, and the method further comprises, after step (e), irradiating the boron.

43. The method of claim 42, wherein the boron addend is labeled with a detectable label and the method further comprises, prior to the step of irradiating the boron, detecting the detectable label to determine the location of the boron addend.

44. The method of claim 1, wherein the therapeutic agent is a beta- or alpha-emitting radioisotope, a drug, a toxin, a boron addend, a vasodilator, a cytokine, a photosensitizer or a radiosensitizer.

45. The method of claim 1, wherein the prodrug is CPT-11 and the therapeutic agent is camptothecin.

46. The method of claim 1, wherein the target site is selected from the group consisting of tumors, infectious lesions, parasitic lesions, fibrin clots, myocardial infarctions, atherosclerotic plaques, non-cancerous cells and damaged normal cells.

47. The method of claim 1, wherein the first binding site specifically binds to a cancer marker selected from the group consisting of MUC1, sTn, Le(y), PMSA, Her2/neu, CD20, GM2 and GD3.

48. The method of claim 1, wherein the first binding site specifically binds to a cancer marker selected from the group-consisting of CSAp and DNA histone.

49. The method of claim 1, wherein the compositions are administered by an intracavitary, intravenous, intraarterial, intrapleural, intrathecal, intralymphatic, intramuscular, intralesional, subcutaneous or catheter perfusion route.

50. The method of claim 1, wherein the patient is a human.

51. A kit for targeting a therapeutic agent to a target site in a patient, comprising, in separate containers:
(a) at least one multispecific targeting protein comprising at least one first binding site which specifically binds to a substance produced by or associated with the target site and present at the target site and at least one second binding site which specifically binds to an epitope on an enzyme, wherein binding between the targeting protein and the enzyme does not interfere with enzyme activity;
(b) optionally, a first clearing composition comprising a clearing agent which clears non-localized targeting protein from circulation;
(c) the enzyme;
(d) optionally, a second clearing composition comprising a clearing agent which clears non-localized targeting protein, non-localized enzyme, or non-localized targeting protein-enzyme conjugate from circulation; and
(e) a serum-soluble prodrug composition, wherein the enzyme of component (c) acts on the prodrug to release a therapeutic agent that is less soluble in serum than the prodrug, and wherein the therapeutic agent partitions out at the target site such that it accretes at the target site to a greater extent than would the prodrug.

52. A sterile injectable preparation for targeting a therapeutic agent to a target site in a patient, comprising:
(a) at least one first sterile injectable solution comprising a multispecific targeting protein comprising at least one first binding site which specifically binds to a substance produced by or associated with the target site and present at the target site and at least one second binding site which specifically binds to an epitope on an enzyme, wherein binding between the targeting protein and the enzyme does not interfere with enzyme activity;
(b) optionally, a second sterile injectable solution comprising a first clearing composition comprising a clearing agent which clears non-localized targeting protein from circulation;
(c) a third sterile injectable preparation comprising the enzyme;
(d) optionally, a fourth sterile injectable preparation comprising a second clearing composition comprising a clearing agent which clears non-localized targeting protein, non-localized enzyme, or non-localized targeting protein-enzyme conjugate from circulation; and
(e) a fifth sterile injectable preparation comprising a serum-soluble prodrug composition, wherein the enzyme of preparation (c) acts on the prodrug to release a therapeutic agent that is less soluble in serum than the prodrug, and wherein the therapeutic agent partitions out at the target site such that it accretes at the target site to a greater extent than would the prodrug.

53. A method for targeting a therapeutic agent to a target site in a patient, comprising:
(a) administering to the patient at least one targeting composition comprising at least one targeting protein-enzyme conjugate comprising a binding site which specifically binds to a substance produced by or associated with the target site and present at the target site, and allowing the conjugate to localize at the target site;
(b) optionally, administering to the patient a clearing composition comprising a clearing agent which clears non-localized targeting protein-enzyme conjugate from circulation; and
(c) administering to the patient at least one serum-soluble prodrug composition, wherein the enzyme of the targeting protein-enzyme conjugate administered in step (a) acts on the prodrug to release a therapeutic agent that is less soluble in serum than the prodrug, and wherein the therapeutic agent partitions out at the target site such that it accretes at the target site to a greater extent than would the prodrug, thereby providing therapeutic agent at the target site.

54. The method of claim 53, wherein the targeting protein-enzyme conjugate comprises a fusion protein of the targeting protein and the enzyme.
